(19) United States
(12) Patent Application Publication
(10) Pub. No.: US 2009/0175973 A1
(43) Pub. Date: Jul. 9, 2009

(54) COFFEE CHERRY COMPOSITIONS AND METHODS FOR THEIR USE IN THE TREATMENT OF DIABETES AND DIABETES RELATED DISORDERS

(76) Inventor: NINA VIKHRIEVA, Moscow (RU)

Correspondence Address: STEMEDICA CELL TECHNOLOGIES, INC, 5375 MIRA SORRENTO PLACE, SUITE 100, SAN DIEGO, CA, 92121, US

(21) Appl. No.: 12/202,951

(22) Filed: Sep. 2, 2008

Related U.S. Application Data
(60) Provisional application No. 60/970,153, filed on Sep. 5, 2007.

(51) Int. Cl. A61K 36/00

(52) U.S. Cl. 424/777

(57) ABSTRACT

Compositions of coffee cherry, methods for their use in the treatment of diabetes and diabetes-related complications, and methods for manufacture of the compositions are disclosed. The compositions find use in the treatment of a variety of diabetes-related conditions including disorders relating to impaired vascular function. Methods for the treatment of macroangiopathies and microangiopathies, including diabetic retinopathy, are disclosed.

This application claims priority to provisional application Ser. No. 60/970,153 filed Sep. 5, 2007, the entire contents of which are incorporated herein in by reference.

### FIELD OF THE INVENTION

The invention is in the field of therapeutic nutraceuticals. In particular, the invention relates to compositions of coffee cherry and methods for their use in the treatment of diabetes and diabetes-related disorders such as diabetic retinopathy.

### BACKGROUND

Coffee cherry is the whole fruit produced by the coffee tree. Coffee cherry is recognized for its nutritional value, particularly its high antioxidant content. Coffee cherry has many beneficial substances if the fruit can be preserved or used in a non-degraded (non-fermented) state. For example, fresh coffee cherry pulp contains high levels of polyphenol antioxidants, and fresh mucilage contains complex polysaccharides and antioxidants. The hull also contains small amounts of polyphenols (less than pulp or green coffee beans) and thus could be used as an additional source for antioxidants.

Diabetes, both Types I and II, lead to a number of complications. Lack of proper glucose metabolism over time can cause heart disease, stroke, kidney disease eye complications, diabetic neuropathy and nerve damage. Diabetes-related eye conditions are particularly troublesome as diabetic retinopathy is the leading cause of blindness of working age individuals in industrialized countries.

Over time, high blood sugar levels can damage the blood vessels that feed the retina of the eye. In nonproliferative diabetic retinopathy (NPDR), an early stage of diabetic eye disease, the blood vessels may leak fluid. This may cause the retina to swell and vision to blur, a condition called diabetic macular edema. In advanced or proliferative diabetic retinopathy (PDR), abnormal new blood vessels grow on the surface of the retina. The abnormal blood vessels don't supply the retina with normal blood flow. In addition, they may eventually pull on the retina and cause it to detach.

Some cases of diabetic retinopathy can be treated with laser surgery. In this procedure, doctors aim a strong beam of light onto the patient's retina to shrink or seal leaking or abnormal vessels. Laser surgery can't restore vision already lost, so early detection is important. In some advanced cases of PDR, a surgeon may remove the vitreous portion of the eye and replace it with a clear solution (called a vitrectomy). These procedures however are invasive and are generally administered only after significant damage to the retina has occurred. What is needed therefore is a non-invasive diabetic retinopathy treatment that addresses the disease before it reaches an advanced state.

### SUMMARY OF THE INVENTION

The inventor has discovered that compositions derived from coffee cherry have a therapeutic benefit in the treatment of diabetes and diabetes-related disorders. The invention provides compositions derived from coffee cherry, and methods for their use in the treatment of diabetes and its associated complications such as diabetic retinopathy.

An object of the invention is to provide a method for treating a patient for diabetes comprising administering to the patient a therapeutically effective amount of coffee cherry.

A further object of the invention is to provide a method for treating a patient for a diabetes-related disorder comprising administering to the patient a composition comprising a therapeutically effective amount of coffee cherry.

A further object of the invention is to provide a method for treating a patient for diabetic retinopathy comprising administering to the patient a composition comprising a therapeutically effective amount of coffee cherry.

A further object of the invention is to provide a composition of coffee cherry effective in the treatment of diabetes and diabetes-related disorders, wherein the composition is optionally supplemented with at least one antioxidant.

A further object of the invention is to provide a composition of coffee cherry effective in the treatment of diabetes and diabetes-related disorders, wherein the composition is supplemented with vitamin C.

### DEFINITIONS

“Coffee cherry” and “cherry” are used interchangeably herein to refer to one or more of the components of the whole fruit produced by the coffee tree (genus coffea, e.g. coffee arabica, or coffee canephora). These components include the stem, exocarp (outer skin), the mesocarp (thin layer of pulp underneath the exocarp), the pharenchyma (lying between the mesocarp and endocarp), the endocarp (which covers the underlying spermoderm), the spermoderm, or “silver skin,” which covers the coffee beans (i.e. seeds), and the beans themselves (see e.g. FIG. 1). The compositions of the invention may be formulated using one or more of these components.

The terms “carrier” and “bulking agent” are used interchangeably herein to refer to any non-toxic material that is suitable for adding volume to the compositions of the invention, and which may be safely administered to a human or lower animal. Examples of carriers include, but are not limited to, starch and starch-containing materials, proteins (e.g. non-fat dry milk), flour, sugar, soybean meal, and maltodextrin.

The term “extract,” as used herein, refers a pharmaceutical preparation of the active molecular constituents of a plant material. Extracts may exist in liquid, gel or solid form (e.g. powders and tablets) and may have varying levels of concentration. Coffee cherry is one non-limiting example of an extract.

“Coffee cherry isolate” as used herein refers to a purified preparation of a molecular constituent of coffee cherry. Such purified isolates are essentially free of other materials meaning that they are least 80 percent pure by weight.

The term “supplement,” as used herein, refers to the addition of a therapeutic material to the composition of the invention. For example, the coffee cherry compositions of the invention may be supplemented with the addition of one or more antioxidants, including, but not limited to, vitamin C. The term “supplement” (e.g. “nutritional supplement”) may also refer to a product that is taken in addition to meals as a means for increasing an individual's nutrient intake. The coffee cherry compositions of the invention are one non-limiting example of a supplement.

“Diabetes-related disorder” means a disorder that is clinically associated with (i.e. results from) the effects of diabetes or pre-diabetes. Examples of diabetes-related disorders include, but are not limited to, heart disease (e.g. atherosclerosis), stroke, diabetic neuropathies, diabetic retinopathy, glaucoma, cataracts, erectile dysfunction, hypoglycemia, kidney disease and gastroparesis. Diabetes-related disorders include skin conditions such as diabetic dermopathy, necrobiosis lipoidica diabeticorum, eruptive xanthomatosis, disseminated granuloma annulare, acanthosis nigricans, allergic reactions, diabetic blisters and digital sclerosis.

“Pre-diabetes” as used herein refers to a condition in which blood glucose levels are higher than normal but do not reach a level high enough for a diagnosis of diabetes. A fasting blood sugar above 100 mg/dL, but less than 125 mg/dL, is one blood sugar range indicative of pre-diabetes.

“Treat,” or “treatment,” as used herein refers to the administration of a therapeutic amount of a coffee cherry composition resulting in the reduction, reversal, delay, or prevention of pre-diabetes, diabetes and/or a diabetes-related condition.

### DETAILED DESCRIPTION

The invention relates to compositions of coffee cherry, to methods for their use in the treatment of diabetes and diabetes-related complications, and to methods for producing therapeutic coffee cherry compositions.

The compositions of the invention may be derived from any combination of the components of coffee cherry provided that the resulting composition is effective in the treatment of diabetes or a diabetes-related condition. The composition may derived from the stem, exocarp, mesocarp, parenchyma, endocarp, spermoderm, seeds and combinations thereof. In some embodiments of the invention, the composition is derived from whole coffee cherry fruit with only the stem and seeds removed. In other embodiments, the composition is derived from the entire coffee cherry fruit.

The compositions of the invention may have varying levels of purity. In some embodiments, the compositions comprise a pure preparation of coffee cherry. As used herein, “pure,” or “purified,” refers to a preparation of coffee cherry that is essentially free (i.e. less than 80% by weight) of other non-coffee cherry constituents. One non-limiting example of a pure coffee cherry composition is one which consists of a fresh preparation of coffee cherry that is free of any other non-coffee cherry constituents. Another example of a pure coffee cherry composition is one consisting of an extract of coffee cherry that is free of other non-coffee cherry constituents such as solvents which may have been used in preparing the extract.

The compositions of the present invention may be derived from coffee cherry extract. Coffee cherry extracts for use with the invention are concentrated preparations of the molecular constituents of coffee cherry. Such extracts may exist in a solid, gel or liquid form and may have varying levels of concentration. The concentration of the extracts for use with the invention will depend upon the level of other constituents present in the composition. For example, the concentration of a coffee cherry extract may be diminished by the presence of one or more solvents that may be used in preparing the extract. The coffee cherry extract's concentration may also be diminished by the presence of carriers, such as for example, liquid or powdered materials that are added to increase the volume of the coffee cherry composition.

In addition to purified compositions of coffee cherry, the invention may embody preparations derived from one or more coffee cherry isolates. The composition of the invention may assume any one or more coffee cherry isolates that provide a therapeutic benefit when administered in the treatment of diabetes or a diabetes-related condition. As used herein, a coffee cherry isolate refers to a purified preparation of a single molecular constituent of coffee cherry. Examples of coffee cherry isolates for use with the invention include, but are not limited to, chlorogenic acid, caffeic acid, ferulic acid, quinic acid, trigonelline, proantocyanidins, gallic, epigallocatechin, trolox, merycetin, cyaniding, sterols, tocopherols, diterpenic alcohols (cafestol, kahweol and kauranic derivatives, and polyphenols. It is contemplated that the invention may be practiced by formulating a composition of one or more of these isolates, or by adding one or more of these isolates to a purified coffee cherry preparation such as a coffee cherry extract. Thus, for example, the composition may comprise coffee cherry extract that is supplemented with chlorogenic acid.

The composition of the invention may assume the form of a fresh preparation of coffee cherry. Such fresh preparations have undergone minimal processing and generally exist in their natural concentration. For example, the composition of the invention may assume fresh preparation of coffee cherry including, but not limited to, juices, mashes, and combinations thereof. Such fresh coffee cherry compositions may be administered alone, or as an additive to a food or beverage.

The inventive composition may assume a formulation that combines coffee cherry with one or more other agents useful in the treatment of diabetes and diabetes-related conditions. These agents may be synthetic in nature, or derived from natural sources including fruits and vegetables.

The compositions of the invention may be derived from any source of coffee cherry that provides a therapeutic benefit in the treatment of diabetes and diabetes-related conditions. Suitable coffee cherry for use with the invention may be obtained, for example, from fresh coffee cherry, dried (e.g. quick-dried) coffee cherry, frozen coffee cherry and combinations thereof.

Coffee cherry suitable for use with the invention may have varying degrees of ripeness. It is contemplated that the compositions of the invention may be prepared using overripe coffee cherry, ripe coffee cherry, semi-ripe coffee cherry, unripe (i.e. green) coffee cherry, and combinations thereof. Where extraction of polyphenols and/or chlorogenic acid from the whole coffee cherry is desired, semi-ripe coffee cherries may be used. On the other hand, where the coffee cherry is used for the production of a coffee cherry tea and flavor and aroma are paramount, almost ripe coffee cherries may be picked. In still further contemplated aspects, unripe coffee cherries, or any reasonable mixtures of varying ripeness degrees may be used. The whole cherry is preferably free of surface defects, including cracks, splits, holes, or other openings. However, while not preferred, ripe coffee cherries, and/or coffee cherries with surface defects may also be used. While not limiting to the inventive subject matter, it is generally preferred that the sub-ripe coffee cherries (or coffee cherry mixtures) are washed with water or other aqueous solution (e.g., diluted hypochlorite solution) to remove soil particles and other debris before processing.

### Making the Composition

The compositions of the invention may be made using any method that produces a preparation of coffee cherry that has a therapeutic effect when administered according to the methods disclosed herein. Without limiting the invention to any particular embodiment, coffee cherry for use with the invention may comprise ripe coffee cherry, sub-ripe coffee cherry, green coffee cherry, and combinations thereof.

The compositions of the invention may be derived using a wet mass, typically wet coffee cherry, as a starting material. According to this embodiment, the mass is dried at low temperature, typically room temperature, to avoid any thermal degradation of the therapeutic components of the material. Preferably, the waste material is initially dried to a low moisture mass such as by centrifugation or lyophilization. The material may also be pressed, such as in a continuous belt filter press to remove most of the water, leaving the pressed materials with a moisture content typically of about 20 wt %. The pressed and at least partially dried raw material is then directly extracted with water or an organic solvent that is partially miscible with water at room or slightly elevated temperature, up to about 100° C. Some organic solvents and water form azeotropic mixtures suitable for azeotropic distillation (azeotropic removal of water). After the desired agents are extracted, the water is removed from the extract, such as by vacuum drying or freeze-drying at a lower temperature, while the organic solvent (if used) is removed, usually by vacuum distillation, at a lower temperature to avoid thermal degradation of any of the extract's ingredients that may be sensitive to elevated temperatures. Specific embodiments for this method of preparing coffee cherry compositions for use with the invention are available in U.S. Pat. No. 6,572,915, the disclosure of which is incorporated by reference.

Further methods for preparing coffee cherry extracts suitable for use with the invention are also disclosed in U.S. Pat. No. 6,676,978 and US Pat. App. No. 20050129827 US Pat. App. No. 20060263508 and PCT App. No. WO2002062159, the disclosures of which are incorporated herein by reference.

Any method of preparing the compositions of the invention may be used provided they render a formulation that is effective in the treatment of diabetes an diabetes-related conditions. In preferred embodiments, methods involving high temperature drying of coffee cherry, or highly reactive solvents, should be avoided.

In some embodiments of the invention, coffee cherry compositions are manufactured using methods that limit the amount of mycotoxin in the final preparation. Fresh ripe and overripe coffee cherry is particularly susceptible to fungal breakdown resulting in coffee preparations that have elevated mycotoxin levels. These mycotoxin levels may be mitigated by selecting non-damaged coffee cherries that are most preferably picked at a sub-ripe stage. Sub-ripe coffee cherries also offer the advantage of increased flavor and aroma, as well as relatively higher levels of polyphenols, polysaccharides, and other nutrients. Mycotoxin levels can also be reduced by subjecting freshly picked coffee cherry to an air drier at about 140° F. until a constant weight is obtained. Such dried preparations of coffee cherry have sustained stability and resistance to fungal breakdown. Detailed methods for preparing coffee cherry compositions that are low in mycotoxin are available in US Pat. App. No. 20060263508, the disclosure of which is incorporated herein by reference.

The coffee cherry compositions of the invention may assume any administration form that permits the compositions to have a therapeutic effect when used in the treatment of diabetes or diabetes-related conditions. The coffee cherry compositions may be administered in liquid or solid form (e.g. powders, pills and capsules). Suitable administration formats for the inventive compositions include, but are not limited to, teas, beverages, edible solids and liquid extracts. Edible solids may be in the form of cereals, breads, tofu, hard candies, gummies, cookies, ice cream, cakes, potato chips, pretzels, snacks (e.g., candy or energy bars), and cheese. The compositions of the invention may also be administered as a fresh preparation of coffee cherry juice.

In aspects of the invention, coffee cherry is administered in a pharmaceutical preparation using a carrier. Such compositions can be practiced with any carrier that is safe and that does not interfere or degrade the therapeutic effectiveness of coffee cherry. Examples of suitable carriers for use with the invention include, but are not limited to, dextrose, mannitol, lactose, lecithin, albumin, sodium glutamate, cysteine hydrochloride, maltitol, maltodextrins, solutions and suspensions thereof, and plant fibers. One skilled in the art will appreciate the range of carriers suitable for use with the invention and may consult publications such as, for example, Remington's Pharmaceutical Sciences, Mack Publishing Go. (A. R. Gennaro edit. 1985), the disclosure of which is incorporated herein by reference.

Coffee cherry compositions for use with the invention may be derived from coffee cherry isolates. Coffee cherry provides a number of therapeutic components with high antioxidant value. These components may be isolated and used alone, or in various combinations, to treat diabetes and diabetes-related conditions including diabetic retinopathy. In some embodiments, the inventive composition combines one or more isolates in a therapeutic formulation. In other embodiments, the composition is supplemented with one or more isolates in combination with a fresh preparation of coffee cherry, in combination with a coffee cherry extract or both.

Coffee cherry isolates for use with the invention may include, but are not limited to, chlorogenic acid, caffeic acid, ferulic acid, quinic acid, trigonelline, proantocyanidins, gallic, epigallocatechin, trolox, merycetin, cyaniding, sterols, tocopherols, diterpenic alcohols (cafestol, kahweol and kauranic derivatives), polyphenols, and combinations thereof.

One skilled in the art will appreciate that methods for obtaining coffee cherry isolates are readily available. These methods will vary and their selection will depend on the particular isolate that is desired. For example, US Pat. App. No. 20060263508, the disclosure of which is incorporated herein by reference, teaches the isolation of highly concentrated or solid polyphenols mixture (e.g., coffee acids, including caffeic, chlorogenic, ferulic acids), an aqueous extract of coffee cherry using basic ion exchange resin. Other methods for obtaining coffee cherry isolates (e.g. polyphenols molecules) are taught by the following references the disclosures of which are incorporated herein by reference: US Pat. App. Nos. 20050266104 and 20010021398; Chandra, A., et al. J. Agric. Food Chem. 41:1062 (1992); Wang, H., et al., J. Agric. Food Chem. 45:2556-2560 (1997); and Arora, A. and G. M. Strasburg, J. Amer. Oil Chem. Soc. 74:1031-1040 (1997).

In addition to the methods disclosed herein, and incorporated by reference, coffee cherry compositions for use with the invention may also be obtained commercially. Such commercial preparations are available sources including, but not limited to, Coffeeberry (VDF FutureCeuticals, Inc., Momence, Ill., USA) and CoffeeBerry (Ekomir, Moscow, Russia).

### Methods of Using the Composition

The coffee cherry compositions of the invention find use in treating type I and type II diabetes, and the complications that are associated with these diseases. “Diabetes-related complication,” “diabetes-related condition,” and “diabetes-related disorder” are used interchangeably herein to refer to a medical disorder that results from the irregular blood glucose levels characteristic of type I and type II diabetes. Diabetes-related complications include, but are not limited to, diabetic retinopathy, heart disease, stroke, kidney disease, eye complications, diabetic neuropathy (e.g. autonomic neuropathy), nerve damage, coronary artery disease (CAD), cerebral vascular disease, peripheral arterial disease, skin disorders including diabetic ulcers (e.g. foot ulcers), erectile dysfunction, kidney disease (nephropathy) gastroparesis, and arteriosclerosis.

In some embodiments, the coffee cherry compositions of the invention are used to treat diabetes-related circulatory disorders, including, but not limited to, macroangiopathy and microangiopathy. Macroangiopathy refers to diabetes-induced damage to the medium and large blood vessels of the body. Microangiopathy refers to diabetes-induced damage to small blood vessels and capillaries which compromises capillary function leading to a shortage of oxygen and nutrients in the tissues, and a leakage of proteins into the tissue spaces. Microangiopathy affects capillaries throughout the body, but damage to the microcirculation of the eyes, kidneys and nerves is responsible for the major clinical manifestations which include retinopathy, nephropathy, neuropathy and diabetic foot. Microangiopathy may also be referred to as a “microperfussion disorder.”

One non-limiting example of a microangiopathy capable of being treated with the coffee cherry compositions of the invention is diabetic retinopathy. Without being limited to any particular theory, diabetic retinopathy develops as balloon-like swelling in the retina's tiny blood vessels lead to the formation of microaneurysms. These blood vessels become blocked depriving portions of the retina of a blood supply. This trauma causes the retina to secrete vascularization signals which result in new, abnormal blood vessels being formed. During this stage, known as proliferative diabetic retinopathy, the abnormal vessels proliferate along the retina and extend to the surface of the vitreous gel that fills the eye. The thin fragile walls of the abnormal vessels eventually leak blood into the vitreous gel causing vision loss and ultimately blindness. In the later phases of the disease, continued abnormal vessel growth and scar tissue may lead to further retinal degeneration including retinal detachment and glaucoma. The compositions of the invention may be used to reduce, prevent or reverse the effects of diabetic retinopathy at all stages of the disorder including mild nonproliferative retinopathy, moderate nonproliferative retinopathy, severe nonproliferative retinopathy, and proliferative retinopathy. Administration of the compositions of the invention may improve the retinal blood supply and visual acuity.

The coffee cherry compositions and methods of the invention may also be used to enhance the overall health of the vascular endothelium in diabetic patients. With diabetes, the endothelial lining of the blood vessels can be damaged resulting in the decreased ability of the vessels to relax or dilate. This leads to impaired regulation of blood flow and consequently a shortage of nutrients and oxygen in affected tissues. Diabetes may also impair the strength of the vessel wall (Br J Ophthalmol. 2000 December; 84(12): 1392-1396). The compositions of the invention may be used to maintain the health of the vascular endothelium in diabetics thereby preventing or prolonging the onset of diabetes-related circulatory disorders.

The invention administers a therapeutically effective amount of coffee cherry in the treatment of diabetes and diabetes-related disorders. As used herein, “a therapeutically effective amount” of coffee cherry refers to any amount of coffee cherry that produces a benefit when administered to a patient suffering from diabetes or a diabetes-related complication. Thus, an amount of coffee cherry is considered “therapeutically effective” if it prevents, reverses or inhibits the onset or progression of diabetes or a diabetes-related complication.

One aspect of the invention relates to the dosage of coffee cherry that is used in the treatment of diabetes and diabetes-related disorders. One skilled in the art will appreciate that different diabetics, and different diabetes-related disorders, will require specific coffee cherry formulations. One non-limiting example of a coffee cherry dosage is a dosage of 60 mg of pure coffee cherry that is administered three times daily. Another non-limiting example of a coffee cherry dosage is a dosage comprising 200 mg of pure coffee cherry that is supplemented with 50 mg of vitamin C. Although specific coffee cherry dosages have been disclosed here, one skilled in the art will appreciate that a wide range of dosages and administration schedules may be used in the treatment of diabetes and diabetes-related complications

### Example 1

### Formulation of Coffee Cherry Composition

CoffeeBerry (Ekomir, Moscow, Russia) a commercially prepared coffee cherry composition was used for the treatment of 32 patients suffering from diabetic retinopathy between stages 1-4. The preparation contained a dosage of 60 mg of coffee cherry formulated as an encapsulated extract.

### Example 2

### Treatment Protocol

The clinical study and treatment involved 33 patients including 18 patients with Diabetes mellitus of type I, and 17 patients with Diabetes mellitus of type II. The age of the patients ranged from 24 to 72 (50.1 on average). The duration of diabetes disease is from 10 to 53 years (17.8 on average).

Study parameters included:
Ophthalmologic status (visual acuity without correction, visual acuity with optimal correction, accommodation volume, the photo picture of eye grounds)
Neurological status (neuropathic, symptomatic scale, modified neuropathic dysfunction scale, electromyoneurographia, stimulated electromyographia)
The dynamics of Diabetes mellitus index (HbA 1, glycemia level on an empty stomach)
The dynamics of antioxidant status index (malonic dialdegid, superoxyddismutase)
The status of lipid metabolism (total cholesterol, triglycerides, LDL)

Treatment with coffee cherry composition from Example 1 was administered by the following regimen: 1 capsule 3 times a day for a 3 month period. Intermediate results were based on data collected during each patient's second visit at the end of month 2 of the treatment.

### Example 3

### Results

The results obtained for the treatment are presented in Table 1. Briefly, the result obtained showed that after two months of treatment the improvement of visual acuity averaged 28.125%. The stabilization of processes in the eye ground was also observed with the quantity of hemorrhage diminished.

| Patient | | | Duration of diabetes | Ophtalm. | Laser |
|---|---|---|---|---|---|
| No. | Sex | Age | disease | Diagnosis | coagulation |
| 1 | M | 0 | 0 | [symbol] P3 | [symbol] K |
| 2 | [symbol] | 53 | 12 | [symbol] P2, MaK | [symbol] K |
| 3 | M | 61 | 12 | [symbol] P3 | [symbol] K |
| 4 | [symbol] | 55 | 4 | [symbol] P3 | [symbol] K |
| 5 | [symbol] | 66 | 17 | [symbol] P3 | [symbol] K |
| 6 | [symbol] | 67 | 18 | [symbol] P2 | [symbol] K |
| 7 | [symbol] | 75 | 21 | [symbol] P1, MaK | |
| 8 | M | 60 | 15 | [symbol] P4 | K |
| 9 | [symbol] | 54 | 23 | [symbol] P3 | [symbol] K |
| 10 | [symbol] | 55 | 13 | [symbol] P, MaK | [symbol] K |
| 11 | [symbol] | 53 | 19 | [symbol] P2 | [symbol] K |
| 12 | [symbol] | 26 | 16 | [symbol] P3 | 1 |
| 13 | [symbol] | 62 | 14 | [symbol] P3 | [symbol] K |
| 14 | M | 37 | 10 | [symbol] P2 | [symbol] K |
| 15 | [symbol] | 31 | 21 | [symbol] P4 | [symbol] K.K |
| 16 | [symbol] | 32 | 15 | [symbol] P3 | [symbol] |
| 17 | [symbol] | 40 | 26 | [symbol] P3 | [symbol] K |
| 18 | M | 70 | 25 | [symbol] P1, MaK | [symbol] K |
| 19 | [symbol] | 72 | 9 | [symbol] P2 | [symbol] K |
| 20 | [symbol] | 64 | 12 | [symbol] P3 | [symbol] K |
| 21 | M | 38 | 20 | [symbol] P3 | [symbol] K |
| 22 | [symbol] | 69 | 34 | [symbol] P1, [symbol] | [symbol] K |
| 23 | [symbol] | 27 | 18 | [symbol] P3 | [symbol] K |
| 24 | [symbol] | 33 | 18 | [symbol] P3 | [symbol] |
| 25 | [symbol] | 24 | 11 | [symbol] P3 | [symbol] K |
| 26 | M | 25 | 0.4 | [symbol] | |
| 27 | [symbol] | 68 | 17 | [symbol] P2, MaK | [symbol] K |
| 28 | M | 59 | 53 | [symbol] P3 | [symbol] K |
| 29 | M | 54 | 19 | [symbol] P3 | [symbol] K |
| 30 | [symbol] | 64 | 15 | [symbol] P3 | [symbol] K |
| 31 | [symbol] | 63 | 17 | [symbol] P1 | [symbol] K |
| 32 | M | 60 | 23 | [symbol] P1 | [symbol] K |
| | | 50.1 | 17.8 | | 1 |
| M-10 | | | | | |
| [symbol] -22 | | | | | |
| [symbol] - | 28.125 | | | | |

| Patient | Glycated | Total | | Glycemia |
|---|---|---|---|---|
| No. | hemoglobin | cholesterol | Triglycerides | level |
| 1 | #DIV/0! | 0 | | 9 |
| 2 | 8.6 | 3.9 | 0.92 | 8 |
| 3 | 9.6 | 5.9 | | 7.8 |
| 4 | 8.6 | 4.8 | | 6.4 |
| 5 | 7.1 | 4.9 | | 6.4 |
| 6 | 7.2 | 6.2 | | 6.6 |
| 7 | 8.6 | 5.8 | | 8 |
| 8 | 8.2 | 6.4 | | 5.8 |
| 9 | 9.2 | 4.9 | | 8 |
| 10 | 7.9 | 5.3 | | 7.5 |
| 11 | 10.2 | 5.8 | | 8.6 |
| 12 | | 5.6 | | 5.7 |
| 13 | 14.8 | 6.5 | | 10 |
| 14 | 10.4 | 5.8 | | 8.7 |
| 15 | 7.4 | 4.9 | | 4.8 |
| 16 | 6.8 | 4.8 | | 6.8 |
| 17 | 7.5 | 4.8 | | 6.4 |
| 18 | 8.4 | 5.7 | | 7 |
| 19 | 9.2 | 5 | 1.3 | 8 |
| 20 | 8.2 | 5.9 | | 7.8 |
| 21 | 8.7 | 5.4 | | 8.3 |
| 22 | 7.4 | 5.6 | | 6.9 |
| 23 | 7.4 | 4.7 | | 7.3 |
| 24 | 9.4 | 4.6 | | 6.4 |
| 25 | 8.4 | 4.8 | | 7 |
| 26 | 4.9 | 3.5 | 0.7 | |
| 27 | 4.6 | 4.9 | | 7.6 |
| 28 | 9.9 | 5.4 | | 8.8 |
| 29 | 6.8 | 4.8 | | 6.4 |
| 30 | 7.5 | 5.8 | | 6.9 |
| 31 | 6.9 | 5.2 | | 7.2 |
| 32 | 6.7 | 4.8 | | 5.7 |
| | [symbol] ( | | | |
| | 8.2 | 5.2 | | |
| M-10 | | | | |
| [symbol] -22 | | | | |
| [symbol] - | | | | |
| | | Visual acuity | | Visual acuity |
| Patient | Visual acuity - | with | Visual acuity - | with |
| No. | left eye | correction | right eye | correction |
| 1 | 0.2465 | 0.2 | 0.294354839 | 1 |
| 2 | 0.6 | 1 | 0.3 | 0.4 |
| 3 | 0 | 0 | 0.2 | 0 |
| 4 | 0.05 | 0 | 0.7 | 0 |
| 5 | 0.1 | 0 | 0.2 | 0 |
| 6 | 0.4 | 0.7 | 0.6 | 0.8 |
| 7 | 0.5 | 0 | 0.2 | 0 |
| 8 | 0.01 | 0 | 0.02 | 0 |
| 9 | 0.7 | 0 | 0.4 | 0 |
| 10 | 0.7 | 0 | 0.3 | 0 |
| 11 | 0.1 | 0 | 0.1 | 0.4 |
| 12 | 0.1 | 0.6 | 0.09 | 0.3 |
| 13 | 0.04 | 0 | 0.02 | 0 |
| 14 | 0.7 | 1 | 0.6 | 0.8 |
| 15 | 0.01 | 0 | 0 | 0 |
| 16 | 0.04 | 0 | 0.005 | 0.05 |
| 17 | 0.2 | 0.8 | 0.2 | 0.7 |
| 18 | 0.2 | 0 | 0.1 | 0.4 |
| 19 | 0.01 | 0 | 0.02 | 0 |
| 20 | 0.01 | 0 | 1 | 0 |
| 21 | 0.1 | 0 | 0.08 | 0 |
| 22 | 0.1 | 0 | 0.2 | 0 |
| 23 | 0.7 | 0.8 | 0.6 | 0.7 |
| 24 | 0.005 | 0 | 0.08 | 0.1 |
| 25 | 0.1 | 0.3 | 0.1 | 0 |
| 26 | 0.1 | 1 | 0.2 | 1 |
| 27 | 0.1 | 0 | 0.1 | 0 |
| 28 | 0.4 | 0 | 0.3 | 0 |
| 29 | 1 | 0 | 0.01 | 0 |
| 30 | 0.02 | 0 | 0.4 | 0 |
| 31 | 1.0 | 0 | 1 | 0 |
| 32 | 0.3 | 0 | 1 | 0 |
| | 0.25 | | 0.3 | |
| M-10 | | | | |
| [symbol] -22 | | | | |
| [symbol] - | | | | |

| | | | | Visual acuity |
|---|---|---|---|---|
| Patient | After 1 | After 2 | Visual acuity - | with |
| No. | month | month | left eye | correction |
| 1 | | 2 | 0.367777778 | 0 |
| 2 | 1 | | 0.7 | 1 |
| 3 | | 2 | 0 | 0 |
| 4 | 1 | 2 | | |
| 5 | | 2 | 0.2 | 0.3 |
| 6 | 1 | | | |
| 7 | 1 | | | |
| 8 | 1 | | | |
| 9 | | 2 | 0.1 | |
| 10 | 1 | | 0.7 | 0 |
| 11 | 1 | | | |
| 12 | 1 | | | |
| 13 | 1 | | 0.04 | 0 |
| 14 | 1 | | 0.7 | 1 |
| 15 | | 2 | 0.01 | 0 |
| 16 | | 2 | 0.06 | 0.01 |
| 17 | | 2 | 0.3 | 0.8 |
| 18 | 1 | | | |
| 19 | 1 | | | |
| 20 | | 2 | 0.01 | 0 |
| 21 | 1 | | | |
| 22 | 1 | | | |
| 23 | 1 | | 0.7 | 0.9 |
| 24 | 1 | | | |
| 25 | 1 | | 0.1 | 0.4 |
| 26 | 1 | | | |
| 27 | | 2 | 0.2 | 0 |
| 28 | 1 | | 0.4 | 0 |
| 29 | 1 | | 1 | 0 |
| 30 | 1 | | | |
| 31 | 1 | | 1 | 0 |
| 32 | 1 | | 0.4 | 0 |
| | | | 0.4 | |
| M-10 | | | | |
| [symbol] -22 | | | | |
| [symbol] - | | | | |

| | | Visual acuity | |
|---|---|---|---|
| Patient | Visual acuity - | with | |
| No. | right eye | correction | Notes |
| 1 | 0.5 | 1 | [symbol] - |
| | | | [symbol] - |
| | | | [symbol] |
| 2 | 0.3 | 0.5 | [symbol] |
| 3 | 0.2 | 0 | |
| 4 | | | |
| 5 | 0.4 | 0 | [symbol] |
| 6 | | | |
| 7 | | | |
| 8 | | | [symbol] |
| 9 | 0.6 | 0 | [symbol] - |
| | | | [symbol] |
| 10 | 0.3 | 0 | |
| 11 | | | |
| 12 | | | |
| 13 | 0.02 | 0 | |
| 14 | 0.7 | 1 | |
| 15 | 0 | 0 | |
| 16 | 0.8 | | [symbol] |
| 17 | 0.2 | 0.7 | [symbol] |
| 18 | | 0 | |
| 19 | | | |
| 20 | 1 | 0 | |
| 21 | | 0 | |
| 22 | | | |
| 23 | 0.6 | 0.8 | [symbol] |
| 24 | | 0 | |
| 25 | 0.1 | 0 | |
| 26 | | | |
| 27 | 0.3 | 0 | [symbol] |
| 28 | 0.3 | 0 | |
| 29 | 0.02 | 0 | [symbol] |
| 30 | | | |
| 31 | 1 | 0 | |
| 32 | 1 | 0 | [symbol] |
| | 0.4 | | |
| M-10 | | | |
| [symbol] -22 | | | |
| [symbol] - | | | |

We claim:

1. A method for treating a patient for diabetes, or a diabetes-related disorder, said method comprising administering to said patient a composition comprising a therapeutically effective amount of coffee cherry.

2. The method of claim 1, wherein said composition is supplemented with at least one antioxidant.

3. The method of claim 2, wherein said at least one antioxidant comprises vitamin C.

4. The method of claim 3, wherein said coffee cherry and said vitamin C are in a ratio of 4 to 1 respectively.

5. The method of claim 3, wherein said coffee cherry is present in an amount of 60 milligrams, and said vitamin C is present in an amount of 50 milligrams.

6. The method of claim 3, wherein said diabetes-related disorder is a metabolic disorder.

7. The method of claim 3, wherein said diabetes-related disorder is selected from the group consisting of diabetic retinopathy, diabetic neuropathy, diabetic nephropathy, microvascular disease, macrovascular disease, diabetic ulceration, a metabolic disorder, and a trophical disorder.

8. The method of claim 3, wherein said administering therapeutically stabilizes said patient's blood sugar level.

9. The method of claim 3, wherein said administering therapeutically stabilizes said patient's A1C hemoglobin level.

10. The method of claim 3, wherein said administering therapeutically stabilizes at least one of said patient's level of triglycerides and said patient's level of low density lipoproteins (LDL).

11. The method of claim 3, wherein said administering normalizes said patient's blood sugar level.

12. The method of claim 1, wherein said coffee cherry is administered according to a route selected from orally, topically, sublingually, intranasally, subdermally, parenterally, intrarectally, intravaginally and combinations thereof.

13. The method of claim 1, wherein said administering comprises using at least one of a solid, liquid, gel, sol, and gas.

14. The method of claim 13, wherein said liquid comprises an alcohol, an oil, or water.

15. The method of claim 13, wherein said liquid comprises a beverage.

16. The method of claim 1, wherein said composition is administered orally using at least one of a gum, a losenge, a liquid, a gel, a gummy, a powder, and a paste.

17. The method of claim 1, wherein said composition comprises a food supplement.

18. The method of claim 3, wherein said treating comprises preventing diabetes or said diabetes-related disorder.

19. The method of claim 3, wherein said treating comprises slowing or stopping the progression of diabetes, or said diabetes-related disorder.

20. The method of claim 3, wherein said treating comprises reversing diabetes, or said diabetes-related disorder.

21. The method of claim 1, wherein said coffee cherry comprises coffee cherry extract.

22. The method of claim 21, wherein said coffee cherry extract comprises modified coffee cherry extract.

23. The method of claim 1, wherein said coffee cherry comprises at least one coffee cherry isolate.

24. The method of claim 23, wherein at least a portion of said at least one coffee cherry isolate is derived synthetically or naturally.

25. A composition comprising coffee cherry wherein said coffee cherry is supplemented with a therapeutically effective amount of at least one anti-oxidant.

26. The composition of claim 25, wherein said at least one antioxidant comprises vitamin C.

27. The composition of claim 26, wherein said coffee cherry and said vitamin C are respectively present in a ratio selected from equal, proportionately greater coffee cherry, proportionately greater vitamin C.

28. The composition of claim 25, wherein said coffee cherry is present in an amount of about 60 milligrams, and said vitamin C is present in an amount of about 50 milligrams.
